(19) United States
(12) Patent Application Publication
(10) Pub. No.: US 2004/0166570 A1
(43) Pub. Date: Aug. 26, 2004

(54) Genes involved in polysaccharide production and utilization thereof

(76) Inventors: Takayuki Asahara, Kawasaki (JP); Hisashi Yasueda, Kawasaki (JP)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/772,271

(22) Filed: Feb. 6, 2004

(30) Foreign Application Priority Data
Feb. 10, 2003 (JP) 2003-32075

(51) Int. Cl. C12P 19/04; C07H 21/04; C12N 15/74; C12Q 1/68; C12N 9/10

(52) U.S. Cl. 435/101; 435/252.3; 435/471; 536/23.2; 435/6; 435/193

(57) ABSTRACT

An ability of a methanol-utilizing bacterium to produce a polysaccharide is improved or suppressed using a DNA encoding a protein selected from the group consisting of:

(A) a protein which has the amino acid sequence of SEQ ID NO: 2;

(B) a variant of a protein which has the amino acid sequence of SEQ ID NO: 2 comprising substitution, deletion, insertion or addition of one or several amino acid residues and has an activity for producing a polysaccharide;

(C) a protein which has the amino acid sequence of SEQ ID NO: 4; and

(D) a variant of a protein which has the amino acid sequence of SEQ ID NO: 4 comprising substitution, deletion, insertion or addition of one or several amino acid residues and has an activity for producing a polysaccharide.

### BACKGROUND OF THE INVENTION

1. Field of the Invention

The present invention relates to a technique useful in the microbial industry and, more particularly, relates to genes involved in polysaccharide production of microorganisms and methods of use. Utilization of the genes improves production of polysaccharides useful in microorganisms on one hand, and on the other hand, suppresses syntheses of unnecessary polysaccharides to improve the production of target substances produced by the microorganisms. As a result, it becomes easier to obtain the target substances.

Utilization of the aforementioned production methods are useful in, in particular, microorganisms utilizing C1 compounds, i.e., compounds having one carbon atom, such as methanol.

2. Brief Description of the Related Art

The prior art shows that polysaccharide production by Methylophilus bacteria, a methanol-utilizing bacterium, in particular, Methylophilus methylotrophus, occurs outside of the cells (J. Gen. Microbiol., 135, pp.2859-2867 (1989). However, structures of genes of Methylophilus bacteria that are involved in the polysaccharide production, have not been previously disclosed.

### SUMMARY OF THE INVENTION

It is an object of the present invention to obtain genes involved in the polysaccharide production from Methylophilus bacteria, and thereby provide means for improving production of polysaccharides from C1 compounds. It is a further object of the present invention to improve the production of target substances by utilizing the genes which suppress syntheses of unnecessary polysaccharides.

It is a further object of the present invention to provide a DNA encoding a protein selected from the group consisting of:

(A) a protein which has the amino acid sequence of SEQ ID NO: 2;

(B) a variant of a protein which has the amino acid sequence of SEQ ID NO: 2 comprising substitution, deletion, insertion or addition of one or several amino acid residues and has an activity for producing a polysaccharide;

(C) a protein which has the amino acid sequence of SEQ ID NO: 4; and

(D) a variant of a protein which has the amino acid sequence of SEQ ID NO: 4 comprising substitution, deletion, insertion or addition of one or several amino acid residues and has an activity for producing a polysaccharide.

It is a further object of the present invention to provide the DNA as described above, wherein said DNA is selected from the group consisting of:

(a) a DNA which has the nucleotide sequence of SEQ ID NO: 1;

(b) a DNA which is hybridizable with a DNA having the nucleotide sequence of SEQ ID NO: 1 or a probe that can be produced from the nucleotide sequence under stringent conditions;

(c) a DNA which has the nucleotide sequence of SEQ ID NO: 3; and

(d) a DNA which is hybridizable with a DNA having the nucleotide sequence of SEQ ID NO: 3 or a probe that can be produced from the nucleotide sequence under stringent conditions.

It is a further object of the present invention to provide the DNA as described above, which is originated from a chromosome of a Methylophilus bacterium.

It is a still further object of the present invention to provide a methanol-utilizing bacterium, into which the DNA as described above has been introduced, and the bacterium has improved ability to produce a polysaccharide.

It is a further object of the present invention to provide the bacterium as described above, which is a Methylophilus bacterium.

It is even a further object of the present invention to provide a method for producing a polysaccharide comprising the steps of

A) culturing the bacterium as described above in a medium containing methanol as a major carbon source, allowing accumulation of the polysaccharide in the medium or cells of the bacterium and

B) collecting the polysaccharide from the medium or the cells.

It is even a further object of the present invention to provide a methanol-utilizing bacterium having an ability to reduce production of a polysaccharide, wherein a gene on the bacterium's chromosome has the same nucleotide sequence as the DNA as described above, or which has homology to the DNA to such an extent that homologous recombination results in disruption of the DNA, thereby suppressing expression of the gene.

It is even a further object of the present invention to provide the bacterium as described above, which is a Methylophilus bacterium.

It is even a further object of the present invention to provide a method for producing a target substance comprising the steps of

A) culturing the bacterium as described above which produces the target substance other than polysaccharide in a medium containing methanol as a major carbon source, allowing accumulation of the target substance in the medium or cells of the bacterium and

B) collecting the target substance from the medium or the cells.

According to the present invention, genes involved in the extracellular polysaccharide production of Methylophilus bacteria are provided. The extracellular polysaccharide production can be increased or decreased using these genes.

### DETAILED DESCRIPTION OF THE INVENTION

The inventors of the present invention found genes involved in the polysaccharide production, i.e., “gtfA” gene and “manC” gene, in the genome in the course of analysis of genes of Methylophilus methylotrophus. Furthermore, they confirmed that amounts of the polysaccharides produced by Methylophilus methylotrophus as a host were reduced by disruption of the genes, and thus accomplished the present invention.

DNA of the present invention

The DNA of the present invention is a DNA encoding a protein selected from the group consisting of:

(A) a protein which has the amino acid sequence of SEQ ID NO: 2;

(B) a variant of a protein which has the amino acid sequence of SEQ ID NO: 2 comprising substitution, deletion, insertion or addition of one or several amino acid residues and has an activity for producing a polysaccharide;

(C) a protein which has the amino acid sequence of SEQ ID NO: 4; and

(D) a protein which has the amino acid sequence of SEQ ID NO: 4 comprising substitution, deletion, insertion or addition of one or several amino acid residues and has an activity for producing a polysaccharide.

Hereinafter, the above-mentioned protein (A) or (B) may be referred to as GtfA, and a DNA encoding the GtfA may be referred to as gtfA. The above-mentioned protein (C) or (D) may be referred to as ManC, and a DNA encoding the ManC may be referred to as manC.

The DNA of the present invention may encode both GtfA and ManC.

The DNA of the present invention can be isolated and obtained from a chromosomal DNA of a Methylophilus bacterium, for example, Methylophilus methylotrophus. A wild-type strain of Methylophilus methylotrophus, the AS1 strain (NCIMB No. 10515), is available form the National Collections of Industrial and Marine Bacteria (Address: NCIMB Lts., Torry Research Station, 135, Abbey Road, Aberdeen AB9 8DG, United Kingdom). Although a typical culture method for this strain is described in the catalogue of NCIMB, it can also be grown in the SEII medium described in the examples section.

The genomic DNA of the AS1 strain can be prepared by a known method, and a commercially available kit for preparing the genome may also be used.

Since the nucleotide sequence of the DNA of the present invention was elucidated by the present invention, it can be obtained by synthesizing primers based on the nucleotide sequence and amplifying the DNA by the polymerase chain reaction (PCR) using a chromosomal DNA of a bacterium such as Methylophilus bacterium as a template. Furthermore, the DNA of the present invention can also be obtained by colony hybridization using a probe prepared based on the aforementioned nucleotide sequence or a partial fragment amplified by PCR as a probe.

Preparation techniques for a genomic DNA library, hybridization, PCR, preparation of plasmid DNA, digestion and ligation of DNA, transformation and so forth used in cloning of the DNA of the present invention are described in Sambrook, J., Fritsch, E. F., Maniatis, T., Molecular Cloning, Cold Spring Harbor Laboratory Press, Third Edition (2001).

Examples of the primers useful for the aforementioned PCR include oligonucleotides of SEQ ID NOS: 5 and 6 for gtfA and oligonucleotides of SEQ ID NOS: 10 and 11 for manC.

The nucleotide sequences of gtfA and manC isolated from the genome of Methylophilus methylotrophus, which were obtained as described above, are shown in SEQ ID NOS: 1 and 3, respectively. The amino acid sequences of GftA and ManC encoded thereby are shown in SEQ ID NOS: 2 and 4, respectively.

As for the amino acid sequences of the aforementioned GtfA and ManC, a known database was searched for amino acid sequences having homology thereto. As a result, it was found that GtfA had 43% homology to a gene product expected to encode glycosyltransferase of Klebsiella pneumoniae (orf-14 in Genbank DB Accession No. 21242). In this search, the homology was examined for the region from position 81 to position 467 of GtfA and the region from position 84 to position 467 of orf-14. Furthermore, ManC showed a 56% homology to the cpsB (manC) gene product of Escherichia coli. The homology was examined between the region from position 1 to position 473 of ManC and the region from position 1 to position 478 of the cpsB product. The homology was calculated as a ratio of identical amino acid residues to the total number of amino acid residues in the region used for comparison.

The DNA of the present invention may encode an amino acid sequence having substitution, deletion, insertion or addition of one or several amino acid residues at one or more positions, so long as the activity of the encoded GtfA or ManC is not substantially diminished. Although the number of “several” amino acid residues referred to herein differs depending on positions or types of amino acid residues in the three-dimensional structures of the proteins, the amino acid sequences may have homology of 70% or more, preferably 80% or more, more preferably 90% or more, most preferably 95% more to the whole amino acid sequence constituting GtfA or ManC and the exhibiting the activity of GtfA or ManC. Specifically, the number of “several” amino acid residues referred to herein may be preferably 2 to 20, more preferably 2 to 10. The aforementioned activities of GtfA and ManC are specifically activities for producing a polysaccharide. In particular, the GtfA activity is the galactosyl-1-phosphate transferase (galactosyl-P—P-undecaprenyl synthetase) activity, which transfers the galactosyl-1-phosphate moiety of GDP-galactose to undecaprenyl phosphate, and the ManC activity refers to the activity of the mannose-1-phosphate guanosyltransferase, which converts mannose-1-phosphate to GDP-mannose.

A DNA encoding a protein substantially identical to GtfA or ManC as described above can be obtained by modifying the nucleotide sequence shown in SEQ ID NO: 1 or 3. For example, site-specific mutagenesis can be employed so that substitution, deletion, insertion or addition of an amino acid residue or residues occur at a specific site. Furthermore, a DNA modified as described above can also be obtained by conventionally-known mutation treatments. Examples of such mutation treatments include a method of treating gtfA or manC before the mutation treatment in vitro with hydroxylamine or the like, a method of treating a microorganism, for example, an Escherichia bacterium, containing gtfA or manC before the mutation treatment with ultraviolet ray irradiation or a mutagenesis agent used in a usual mutation treatment such as N-methyl-N′-nitro-N-nitrosoguanidine (NTG) or EMS.

The substitution, deletion, insertion, addition, inversion or the like of nucleotides described above also may include naturally occurring mutations or variations on the basis of, for example, individual difference or difference in species of microorganisms that contain gtfA or manC.

A DNA encoding a protein substantially identical to GtfA or ManC can be obtained by expressing a DNA including any of the aforementioned mutations in a suitable cell and examining the activity of the expression product. Examples of DNA encoding a protein substantially identical to GtfA or ManC include DNA which is hybridizable with nucleotide sequence of the nucleotide numbers 4 to 1401 in the nucleotide sequence of SEQ ID NO: 1 or a probe prepared from the nucleotide sequence for gtfA, or the nucleotide numbers 4 to 410 in the nucleotide sequence of SEQ ID NO: 3 or a probe prepared from the nucleotide sequence for manC, under stringent conditions, and which encodes a protein having the activity of GtfA or ManC.

The “stringent conditions” referred to herein include a condition under which a so-called specific hybrid is formed, and non-specific hybrid is not formed. It is difficult to clearly express this condition using any numerical value. However, for example, the stringent conditions include a condition whereby DNAs having high homology, for example, DNAs having homology of 70% or more, preferably 80% or more, more preferably 90% or more, most preferably 95% or more, are hybridized with each other, whereas DNAs having homology lower than the above do not hybridize with each other. Alternatively, the stringent conditions include conditions whereby DNAs hybridize with each other at a salt concentration upon ordinary conditions of washing in Southern hybridization, i.e., 1×SSC, 0.1% SDS, preferably 0.1×SSC, 0.1% SDS, at 60° C.

A partial sequence of gtfA can also be used for gtfA, or a partial sequence of manC can also be used for manC as the probe. Probes can be generated by PCR using oligonucleotides based on the nucleotide sequence of each gene as primers, and a DNA fragment containing each gene as a template using a method well-known to those skilled in the art. When a DNA fragment in a length of about 300 bp is used as the probe, the washing conditions of hybridization can be, for example, 50° C., 2×SSC and 0.1% SDS.

The activity of GtfA may be measured by the method of Jiang, X.-M. et al. (described in Molecular Microbiology, vol. 5, pp.695-713). Examples of the method for measuring the activity of ManC include, for example, the method of Cabib, E. & Leloir, L. F. (see Journal of Biological Chemistry, vol. 231, pp.259-275).

<1> Methanol-Utilizing Bacterium of the Present Invention

The bacterium according to the first embodiment of the present invention is a methanol-utilizing bacterium which is introduced with gtfA or manC and has improved the ability to produce a polysaccharide. A bacterium introduced with both of gtfA and manC is also encompassed by the present invention.

The bacterium according to the second embodiment of the present invention is a methanol-utilizing bacterium in which gtfA or manC, or a gene having homology to either one of them in such a degree that homologous recombination can be caused with either one of the genes is disrupted, thereby expression of the gene is suppressed, and an ability to produce a polysaccharide is reduced, and which has an ability to produce a target substance other than polysaccharide. A bacterium in which both of gtfA and manC or homologues of both gtfA and manC are disrupted is also encompassed by the present invention.

Methanol-utilizing bacterium to which the present invention can be applied is not particularly limited, so long as gtfA or manC can function in the bacterium, or the bacterium has gtfA, manC or a homologue of either one of them. Specific examples include Methylophilus bacteria such as Methylophilus methylotrophus, Methylobacillus bacteria such as Methylobacillus glycogenes and Methylobacillus flagellatum and Methylobacterium bacteria such as Methylobacterium extorquens. Among these, Methylophilus bacteria are preferred, and Methylophilus methylotrophus is particularly preferred.

The bacterium according to the first embodiment of the present invention can be constructed by introducing gtfa or manC into a methanol-utilizing bacterium in a state that GtfA or ManC encoded thereby can be expressed. The gene gtfA or manC can be introduced into a methanol-utilizing bacterium by, for example, ligating the gene fragment containing gtfA or manC with a vector functioning in the methanol-utilizing bacterium, preferably a multi-copy vector, to produce a recombinant DNA, and transforming the methanol-utilizing bacterium with the recombinant DNA. Any method can be used to introduce the recombinant DNA into the methanol-utilizing bacterium, so long as it provides sufficient transformation efficiency. For example, electroporation can be encompassed (Canadian Journal of Microbiology, 43, p.197 (1997)). In addition, it is possible to incorporate gtfA or manC into a host chromosome by a method using transduction, transposon (Berg, D. E. and Berg, C. M., Bio/Technol., 1, p.417, 1983), Mu phage, (Japanese Patent Laid-open (Kokai) No. 2-109985) or homologous recombination (Experiments in Molecular Genetics, Cold Spring Harbor Lab. (1972)). Furthermore, a promoter that functions in a methanol-utilizing bacterium can be ligated to the upstream or the gtfA or manC, as required.

The vectors as described above include, specifically, a vector that can autonomously replicate in a host methanol-utilizing bacterium, for example, Methylophilus methylotrophus. Examples include RSF1010, which is a wide host range vector, and derivatives thereof, for example, pAYC32 (Chistorerdov, A. Y., Tsygankov, Y. D., Plasmid, 16, pp.161-167 (1986)), pMFY42 (Gene, 44, p.53 (1990)), pBBR1 and derivatives thereof (Kovach, M. E., et al., Gene, 166, pp.175-176 (1995)), pRK310 and derivatives thereof (Edts. Murrell, J. C., and Dalton, H., Methane and methanol utilizers, Plenum Press, pp.183-206 (1992)) and so forth.

The bacterium according to the second embodiment of the present invention can be constructed by disrupting gtfA or manC, or a homologue thereof having homology to either one of them in such a degree that homologous recombination should be caused with either one of them (hereinafter, also simply referred to as “gtfA or manC”) on a chromosome so that the gene product thereof should not normally function. The aforementioned homology in such a degree that homologous recombination should be caused is preferably 90% or more, more preferably 95% or more, particularly preferably 99% or more.

Examples of the method for obtaining a methanol-utilizing bacterium in which gtfA or manC is disrupted include a method of treating a methanol-utilizing bacterium with ultraviolet irradiation or a mutagenesis agent used for a conventional mutation treatment such as N-methyl-N′-nitro-N-nitrosoguanidine (NTG) or EMS and selecting a mutant strain showing reduced activity of GtfA or ManC.

Furthermore, gtfA or manC on a chromosome can also be disrupted by gene substitution using homologous recombination (Experiments in Molecular Genetics, Cold Spring Harbor Laboratory Press (1972); Matsuyama, S. & Mizushima, S., J. Bacteriol., 162, 1196 (1985)) as described in the examples section. The ability to cause homologous recombination is a property generally possessed by bacteria, and the inventors of the present invention found that gene substitution utilizing homologous recombination was also possible in Methylophilus bacteria. Specifically, a methanol-utilizing bacterium is transformed with a DNA containing gtfA or manC modified so as not to produce GtfA or ManC which normally functions (deletion-type gene) to cause recombination between the deletion type-gene and gtfA or manC on the chromosome. Thereafter, if recombination is caused again at a site on the chromosome to which the plasmid is incorporated, the plasmid is eliminated from the chromosome. At this time, depending on the site where the recombination occurs, the deletion-type gene may be left on the chromosome, and the native gene may be eliminated from the chromosome along with the plasmid, or the native gene may be left on the chromosome, and the deletion-type gene may be eliminated from the chromosome along with the plasmid. By selecting a strain in which the former case occurred, a strain in which the deletion-type gene substitutes for the native gene on the chromosome can be obtained.

Furthermore, the inventors of the present invention also found that, in Methylophilus methylotrophus, introduction of a gene homologous to a target gene on a chromosome in the form of a linear DNA fragment caused homologous recombination between the target gene on the chromosome and the homologous gene on the introduced linear DNA fragment in the cell, and thereby gene substitution could be attained, and such a technique can also be applied. An example of gene substitution performed using above-mentioned technique is described in the examples section.

Examples of the aforementioned deletion-type gene include genes in which substitution, deletion, insertion, addition or inversion of one or more nucleotides is caused in the nucleotide sequence of coding region and thereby specific activity of the encoded protein is reduced or eliminated as well as genes of which internal portion or terminal portion of the coding region is deleted, genes of which coding region is inserted with another sequence and so forth. Examples of the other sequence include marker genes such as the kanamycin resistance gene.

Expression of gtfA or manC on a chromosome can also be reduced or eliminated by introducing substitution, deletion, insertion, addition or inversion of one or several nucleotides into a promoter sequence of the gene to reduce the promoter activity and thereby suppressing a transcprition of the gene (see Rosenberg, M. & Court, D., Ann. Rev. Genetics, 13, p.319 (1979); Youderian, P., Bouvier, S. & Susskind, M., Cell, 30, pp.843-853 (1982)).

Furthermore, expressions of these genes can also be suppressed at a translation level by introducing substitution, deletion, insertion, addition or inversion of one or several nucleotides into a region between the SD sequence and the initiation codon (see Dunn, J. J., Buzash-Pollert, E. & Studier, F. W., Proc. Natl. Acad. Sci. U.S.A., 75, p.2743 (1978)).

The modification of a region between a promoter or SD sequence and an initiation codon described above can be performed in the same manner as that for the aforementioned gene substitution.

Methods for introducing substitution, deletion, insertion, addition or inversion of nucleotides into a gene include the site-specific mutagenesis (Kramer, W. & Frits, H. J., Methods in Enzymology, 154, 350 (1987)) and a treatment with a chemical agent such as sodium hyposulfite or hydroxylamine (Shortle, D. and Nathans, D., Proc. Natl. Acad. Sci. U.S.A., 75, 270 (1978)).

Site-specific mutagenesis is a method using a synthetic oligonucleotides, which can introduce arbitrary substitution, deletion, insertion, addition or inversion into specific base pairs. In order to utilize this method, a plasmid harboring a desired gene that is cloned and has a determined DNA nucleotide sequence is first denatured to prepare a single strand. Then, a synthetic oligonucleotide complementary to a region where a mutation is desired to be introduced is synthesized. In this synthesis, the sequence of the synthetic oligonucleotide is not prepared as a completely complementary sequence, but is made to include substitution, deletion, insertion, addition or inversion of an arbitrary nucleotide or nucleotides. Thereafter, the single-stranded DNA and the synthetic oligonucleotide including substitution, deletion, insertion, addition or inversion of an arbitrary nucleotide or nucleotides are annealed, and a complete double-stranded plasmid is synthesized using Klenow fragment of DNA polymerase I and T4 ligase and introduced into competent cells of Escherichia coli. Some of the transformants obtained as described above would have a plasmid containing the desired gene in which substitution, deletion, insertion, addition or inversion of an arbitrary nucleotide or nucleotide is fixed.

The recombinant PCR method (PCR Technology, Stockton Press (1989)) can be employed as a similar method that enables introduction of mutation and thereby modification or disruption of the gene.

The bacterium according to the second embodiment of the present invention is preferably a bacterium having an ability to produce a target substance other than polysaccharide, for example, amino acids such as L-lysine, nucleic acids, vitamins, proteins such as enzymes and so forth.

As such a bacterium as described above, a Methylophilus bacterium having an ability to produce L-lysine, for example, a Methylophilus methylotrophus strain, can be obtained by subjecting such a strain not having an ability to produce L-lysine or having a reduced ability to produce L-lysine to a mutagenesis treatment to impart to it resistance to an L-lysine analogue such as S-(2-aminoethyl)-L-cysteine (hereinafter referred to as “AEC”). Examples of the method for the mutagenesis treatment include methods of treating cells with a chemical mutagenesis agent such as NTG or EMS or with irradiation of ultraviolet ray or radial ray or the like. Specific examples of such a strain include Methylophilus methylotrophus AJ13608. This strain was bred by imparting the AEC resistance to the Methylophilus methylotrophus AS 1 strain. The Methylophilus methylotrophus AJ13608 was deposited at the National Institute of Bioscience and Human-Technology, Agency of Industrial Science and Technology (currently, National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Jun. 10, 1999 and received an accession number of FERM P-17416. Then, the deposit was converted to an international deposit under the provisions of the Budapest Treaty on Mar. 31, 2000 and received an accession number of FERM BP-7112.

A Methylophilus methylotrophus strain having an ability to produce L-lysine can also be bred by introducing a DNA carrying genetic information involved in the biosynthesis of L-lysine or enhancing the expression of the gene using a genetic recombination technique. The gene to be introduced is a gene encoding an enzyme of the biosynthetic pathway of L-lysine such as dihydrodipicolinate synthase and succinyl diaminopimelate transaminase. In the case of a gene of enzyme suffering from feedback inhibition by L-lysine such as dihydrodipicolinate synthase, it is desirable to use a mutant gene encoding the enzyme of which inhibition is desensitized. Furthermore, it is also considered effective to introduce a secretion carrier of L-lysine such as the lysE gene of Corynebacteirum glutamicum.

A desired gene can be introduced into a methanol-utilizing bacterium in a manner similar to that used for the introduction of gtfA and manC as described above.

A methanol-utilizing bacterium having an ability to produce a target substance and having a disrupted gtfA or manC can be obtained by imparting an ability to produce a target substance to a methanol-utilizing bacterium having a disruptd gtfA or manC. Alternatively, such a bacterium as described above can also be obtained by disrupting gtfA or manC of a Methylophilus bacterium having an ability to produce a target substance.

<3> Method for Producing Polysaccharide or Objective Substance

The bacterium according to the first embodiment of the present invention is introduced with gtfA or manC, and its activity of GtfA or ManC is enhanced. Therefore, the polysaccharide can be efficiently produced by culturing this bacterium in a medium containing methanol as a major carbon source to produce and accumulate a polysaccharide in the medium or cells of the bacterium and collecting the polysaccharide from the medium or cells.

In the bacterium according to the second embodiment of the present invention, gtfA or manC is disrupted, and the ability to produce a polysaccharide, especially a polysaccharide that is secreted to the outside of cells, is reduced. Therefore, when this bacterium is cultured in a medium to produce and accumulate a polysaccharide in the medium or cells of the bacterium, a reduced amount of polysaccharide components can be produced in the medium or cells.

Polysaccharides have industrial applications as gelling agents, thickening stabilizers and so forth, and methods for producing them at a low cost are expected. From this point of view, the bacterium according to the first embodiment of the present invention is useful.

On the other hand, when useful substances such as amino acids, nucleic acids, vitamins, enzymes and proteins are produced as target substances using a methanol-utilizing bacterium, polysaccharides by-produced by the bacterium are unnecessary products. Therefore, it is considered that if by-production of polysaccharides is reduced, energy and carbon that should be consumed for the production of the by-products comes to be effectively utilized for an intended objective product, and thus productivity and yield of the target product are improved. Therefore, the reduction of the by-products is important for industrial applications. Moreover, in case that cells are removed from culture broth by centrifugation or the like, if a bacterium produces polysaccharides in large amounts, it may become difficult to precipitate the cells because the polysaccharides inhibit it. However, by reducing production amounts of polysaccharides, it becomes possible to quickly precipitate the cells. Therefore, the bacterium according to the second embodiment of the present invention is useful for separation of cells from culture broth or obtaining a target substance from culture broth.

Examples of the aforementioned polysaccharides include xanthane gum and so forth.

The medium used for culturing the methanol-utilizing bacteria is a typcial medium that contains a carbon source, nitrogen source, inorganic ions and other organic trace nutrients as required. The major carbon source is methanol. However, sugars such as glucose, lactose, galactose, fructose and starch hydrolysate, alcohols such as glycerol and sorbitol, and organic acids such as fumaric acid, citric acid, succinic acid and pyruvic acid may be used together. The expression “methanol is used as a major carbon source” means that methanol accounts for 50% (w/w) or more, preferably 80% (w/w) or more, of the total carbon source. When methanol is used as a major carbon source, the concentration thereof is usually 0.001% to 4% (w/v), preferably 0.1% to 2% (w/v). Furthermore, when glucose or the like is added, the concentration thereof is usually 0.1% to 3% (w/w), preferably 0.1% to 1% (w/v).

As the nitrogen source, inorganic ammonium salts such as ammonium sulfate, ammonium chloride and ammonium phosphate, organic nitrogen source such as soybean hydrolysate, ammonia gas, aqueous ammonia and so forth can be used.

As the inorganic ions (or sources thereof), small amounts of potassium phosphate, magnesium sulfate, iron ions, manganese ions and so forth are added to the medium. As the organic trace nutrients, vitamin B₁, yeast extract and so forth may be added to the medium in suitable amounts.

The culture is preferably performed for about 16 to 72 hours under an aerobic condition. The culture temperature is controlled to be between 25° C. to 45° C., and pH is controlled to be between 5 to 8 during the culture. Inorganic or organic acidic or alkaline substances, ammonia gas and so forth can be used to adjust to pH.

After completion of the culture, the amount of polysaccharide components in fermentation broth can be measured by a known method, for example, the phenol/sulfuric acid method (Hodge, J. E., Hofreiter, B. T., Methods in Carbohydrate Chemistry, ed. by Whistler, R. L., Wolfrom, M. L., Academic Press, New York, vol. 1, p.388 (1962)).

A polysaccharide or a target substance can be collected by a known method. For example, amino acids such as L-lysine can be appropriately collected by typical methods using ion exchange resins, precipitation and other known methods in combination.

### EXAMPLES

Hereinafter, the present invention is explained more specifically with reference to the following non-limiting examples.

### Example 1

### Acquisition of gtfA (glycosyltransferase) Gene

The Methylophilus methylotrophus AS1 strain (NCIMB No. 10515) was inoculated into 50 mL of the SEII medium (composition: 1.9 g/L of K₂HPO₄, 5.0 g/L of (NH₄)₂SO₄, 1.56 g/L of NaH₂PO₄.2H₂O, 0.2 g/L of MgSO₄.7H₂O, 0.72 mg/L of CaCl₂.6H₂O, 5 μg/L of CuSO₄.5H₂O, 25 μg/L of MnSO₄.5H₂O, 23 μg/L of ZnSO₄.7H₂O, 9.7 mg/L of FeCl₃.6H₂O, 1% (v/v) of methanol) and cultured overnight at 37° C. with shaking. Then, the medium was centrifuged to collect the cells. A chromosomal DNA was purified from the obtained cells using a commercially available kit (Genomic DNA Purification Kit (produced by Edge Biosystems)).

Then, PCR was performed using the obtained genomic DNA (0.05 μg) as a template and DNA primers MgtfA-F1 (SEQ ID NO: 5) and MgtfA-R1 (SEQ ID NO: 6) as primers. The reaction conditions were 94° C. for 10 seconds for denaturation, 50° C. for 30 seconds for annealing and 70° C. for 4 minutes for extension reaction (28 cycles). A commercially available kit (Pyrobest Taq (Takara Bio Inc.)) was used for PCR according to the attached protocol. As a result, a DNA fragment of about 3.8 kbp was amplified. This fragment was digested with the restriction enzyme PstI to obtain a DNA fragment of 2.2 kbp.

Separately, a plasmid vector, pBluescript SK− (Stratagene), was digested with the restriction enzyme PstI to obtain a DNA fragment. Then both DNA fragments as described above were ligated using Ligation Kit (Takara Bio Inc.) to prepare pBS-mGtfA1. In this plasmid, the direction of the gtfA gene was the same as the transcription direction from the lac promoter.

The nucleotide sequence of the DNA fragment cloned as described above was determined in a conventional manner. The sequence is shown in SEQ ID NO: 1, and the amino acid sequence encoded thereby is shown in SEQ ID NO: 2. When amino acid sequence database was searched for amino acid sequences having homology to the above amino acid sequence, the glycosyltransferase of Klebsiella pneumoniae was retrieved. Therefore, the gene of SEQ ID NO: 1 was designated as gtfA.

### Example 2

### Disruption of gtfA Gene in Methylophilus methylotrophus and Effect Thereof

First, restriction enzyme recognition sites existing on the both sides of the Km^{R} (kanamycin resistance) gene region of the plasmid pUC4K (Amersham Biosciences) was partially modified. That is, pUC4K was digested with restriction enzymes EcoRI and SalI, and the digestion ends were blunt-ended. Then, to prepare pUC4K2 the Km^{R} gene DNA fragment and the DNA fragment carrying the replication initiation region (Ori) were ligated using Ligation Kit (Takara Shuzo). That is, pUC4K2 corresponds to pUC4K with restriction enzyme sites EcoRI, BamHI and SalI deleted.

Km4-F2 (SEQ ID NO: 7) and Km4-R2 (SEQ ID NO: 8) were prepared as DNA primers for PCR and used together with pUC4K2 as a template DNA to perform PCR (conditions: 94° C. for 10 seconds for denaturation, 50° C. for 30 second for annealing and 70° C. for 1.5 minutes for extension reaction, 28 cycles) to amplify the DNA fragment carrying the Km^{R} gene. Furthermore, both ends of the amplified DNA were blunt-ended using BKL Kit (Takara Bio Inc.).

Then, pBS-mGtfA1 obtained in Example 1 was digested with the restriction enzymes EcoT141 and MluI and then blunt-ended to prepare a DNA fragment. This DNA fragment and the aforementioned Km^{R} gene DNA fragment were ligated using Ligation Kit to prepare pBS-MgtfA-Δ.

The plasmid pBS-MgtfA-Δ was digested with the restriction enzymes BamHI and SalI to fragment the region containing the gtfA gene interrupted with the kanamycin resistance gene (gtfA::KmR). This fragment was concentrated by ethanol precipitation, further subjected to a desalting treatment and used as a sample of DNA fragment to be introduced by electroporation.

The Methylophilus methylotrophus AS1 strain was cultured at 37° C. for 16 hours with shaking in the SEII liquid medium (methanol concentration: 0.5% (v/v)), and 20 ml of the culture broth was centrifuged at 10,000 rpm for 10 minutes to collect the cells. 1 mM HEPES buffer (pH 7.2, 20 ml) was added to the cells to suspend the cell in the buffer, and the suspension was centrifuged. This operation was performed twice, and 1 ml of the same buffer was finally added to the cells to prepare a cell suspension and used as electro cells for electroporation.

About 1 μg of the aforementioned DNA fragment containing the gtfA gene interrupted with the kanamycin resistance gene (gtfA::KmR) was added to 100 μl of the electro cells, and electric pulses were applied with the conditions of 18.5 kV/cm, 25 μF and 200 ω to perform electroporation and thereby introduce the DNA fragment into the cells. This cell suspension was immediately added to the SEII liquid medium and cultured at 37° C. for 3 hours. Then, this culture broth was applied to the SEII+Km agar medium (SEII medium containing 20 μg/ml of kanamycin and 1.5% (w/v) of agar) and the cells were cultured at 37° C. for three days. As a result, about 100 transformants of Km^{R} were obtained. Among these, six strains were selected, and genomic DNA of each strain was used as a template to perform PCR (reaction conditions were 94° C. for 10 seconds for denaturation, 50° C. for 30 second for annealing and 72° C. for 4 minutes for extension reaction, 30 cycles) and thereby investigate the structure of the gtfA gene region of each candidate strain. The DNA primers used for PCR were MgtfA-F 1 (SEQ ID NO: 5), MgtfA-R1 (SEQ ID NO: 6) and Km4-R1 (SEQ ID NO: 9). As a result, a DNA fragment having a size of 4,100 bp could be amplified with the combination of MgtfA-F1 and MgtfA-R2, and a DNA fragment having a size of 2,900 bp could be amplified with the combination of MgtfA-F1 and Km4-R1, as expected. Thus, a strain deficient in the gtfA gene, which was the target gene of the disruption, could be obtained.

Then, it was investigated whether the production amount of polysaccharide components produced by the cells would be changed by this genetic deficiency. The AS1 strain and a candidate strain for the gtfA gene deficiency were each applied to the SEII agar medium and cultured overnight at 37° C. Then, the cells on about 3 cm² of the medium surface were scraped, inoculated to the SEII production medium (20 ml) and cultured at 37° C. for 35 hours with shaking. After completion of the culture, the cells were removed by centrifugation, and the supernatant was used as a sample for measurement of the amount of extracellular polysaccharides.

For measuring the amount of extracellular polysaccharides, the phenol/sulfuric acid method was used (Dubois, M., Giles, K. A., Hamilton, J. K, Rebers, P. A. and Smith, F., Colorimetric method for determination of sugars and related substances, Anal. Chem., 28:350-356, 1956), which is one of the colorimetric measurement methods applied to neutral saccharides, especially hexoses. Specifically, 0.2 mL of 5% phenol solution was added to 0.2 mL of a sample and mixed. Subsequently, 1 mL of concentrated sulfuric acid was quickly added to the mixture so that the sulfuric acid should be directly added dropwise to the liquid surface, and left for 10 minutes. Then, the mixture was stirred again and left on a water bath at 25° C. for 20 minutes, and the absorbance was measured at 490 nm using an absorptiometer (Hitachi U-2000).

As a result, the amount of extracellular polysaccharides given by the AS1 strain was 226 mg/L, whereas that given by the candidate strain for gtfA gene deficiency was 98 mg/L. Thus, it was found that the amount was reduced to approximately half in the candidate strain, and it was found that the obtained strain was a polysaccharide production-suppressed strain also for the phenotype.

### Example 3

### Acquisition of manC (cpsB) (phosphomannose isomerase/mannose-1-phosphate guanylyltransferase) Gene

PCR was performed using genomic DNA (0.05 μg) of the Methylophilus methylotrophus AS1 strain as a template and the DNA primers mManC-F1 (SEQ ID NO: 10) and mManC-R1 (SEQ ID NO: 11). The conditions were 94° C. for 10 seconds for denaturation, 50° C. for 30 seconds for annealing and 70° C. for 4 minutes for extension reaction (28 cycles). PCR was performed using a commercially available kit, Pyrobest Taq (Takara Bio Inc.) according to the attached protocol. As a result, a DNA fragment of about 1,460 bp could be amplified. This fragment was digested with the restriction enzyme BamHI to obtain a DNA fragment of about 1.46 kbp.

Separately, a plasmid vector, pBR322 (Takara Bio Inc.), was digested with the restriction enzyme BamHI, and the 5′ phosphate of the digested end was dephosphrylated. These two DNA fragments were ligated using Ligation Kit (Takara Bio Inc.) to construct pBS-MmanC. In this plasmid, the direction of the manC gene was the same as the transcription direction of the Amp (ampicillin) resistance gene.

The nucleotide sequence of the obtained DNA fragment was determined in a conventional manner. The sequence is shown in SEQ ID NO: 3, and the amino acid sequence encoded thereby is shown in SEQ ID NO: 4. When amino acid sequence database was searched for amino acid sequences having homology to the above amino acid sequence, manC (cpsB) of Escherichia coli was retrieved. Therefore, the gene of SEQ ID NO: 4 was designated as manC.

### Example 4

### Disruption of manC Gene and Effect Thereof

PCR was performed using Km4-F2 (SEQ ID NO: 7) and Km4-R2 (SEQ ID NO: 8) as DNA primers and pUC4K2 as a template DNA (conditions: 94° C. for 10 seconds for denaturation, 50° C. for 30 second for annealing and 70° C. for 1.5 minutes for extension reaction, 28 cycles) to amplify the DNA fragment carrying the Km^{R} gene. Both ends of the amplified DNA were blunt-ended using BKL Kit (Takara Bio Inc.) to prepare a DNA fragment carrying the Km^{R} gene (1.3 kb).

Then, pBS-MmanC obtained in Example 3 was digested with the restriction enzyme KpnI and blunt-ended, and the 5′ phosphate of the digested ends were dephosphrylated. To prepare pBS-MmanC-Δ, this DNA fragment and the aforementioned DNA fragment carrying the Km^{R} gene were ligated using Ligation Kit.

The plasmid pBS-MmanC-Δ was digested with the restriction enzyme BamHI to excise the region containing the manC gene interrupted with the kanamycin resistance gene (manC::Km^{R}). This fragment was concentrated by ethanol precipitation, further subjected to a desalting treatment and used as a sample of DNA fragment to be introduced by electroporation.

Then, the aforementioned DNA sample was introduced into the AS1 strain by electroporation in the same manner as that of Examples 1 and 2 to obtain transformants. About 100 strains could be obtained as Km^{R} strains. Among these, six strains were selected, and genomic DNA of each was used as a template to perform PCR (reaction conditions were 94° C. for 10 seconds for denaturation, 50° C. for 30 second for annealing and 72° C. for 4 minutes for extension reaction, 30 cycles) and thereby investigate the structure of the manC gene region of each candidate strain. The DNA primers used for PCR were MmanC-F2 (SEQ ID NO: 12) and MmanC-R2 (SEQ ID NO: 13). As a result, a DNA fragment having a size of 3,900 bp was amplified with the combination of MmanC-F2 and MmanC-R2 as expected, and thus a strain deficient in the manC gene, which was the target gene of the disruption, was obtained.

Then, it was investigated whether the production amount of polysaccharide components produced by the cells would be changed by this genetic deficiency. The phenol/sulfuric acid method was used as in Example 2.

The AS1 strain and the candidate strain for the manC gene deficiency were each applied to the SEII agar medium and cultured overnight at 37° C. Then, the cells on about 3 cm² of the medium surface were scraped, inoculated into the SEII production medium (20 ml) and cultured at 37° C. for 45 hours with shaking. After completion of the culture, the cells were removed by centrifugation, and the supernatant was used as a sample for measurement of the amount of extracellular polysaccharides.

As a result, the amount of extracellular polysaccharides given by the AS1 strain was 475 mg/L, whereas that given by the candidate strain for manC gene deficiency was 308 mg/L. Thus, it was found that the amount of extracellular polysaccharides was reduced in the candidate strain, and it was suggested that the obtained strain was a manC-disrupted strain also for the phenotype.

As described above, disruption of the manC gene of Methylophilus methylotrophus by a linear DNA was confirmed.

What is claimed is:

1. An isolated and purified DNA encoding a protein selected from the group consisting of:
(A) a protein which has the amino acid sequence of SEQ ID NO: 2;
(B) a variant of a protein which has the amino acid sequence of SEQ ID NO: 2 comprising substitution, deletion, insertion or addition of one or several amino acid residues and has an activity for producing a polysaccharide;
(C) a protein which has the amino acid sequence of SEQ ID NO: 4; and
(D) a variant of a protein which has the amino acid sequence of SEQ ID NO: 4 comprising substitution, deletion, insertion or addition of one or several amino acid residues and has an activity for producing a polysaccharide.

2. The DNA according to claim 1, wherein said DNA is selected from the group consisting of:
(a) a DNA which has the nucleotide sequence of SEQ ID NO: 1;
(b) a DNA which is hybridizable with a DNA having the nucleotide sequence of SEQ ID NO: 1 or a probe that can be produced from said nucleotide sequence under stringent conditions;
(c) a DNA which has the nucleotide sequence of SEQ ID NO: 3; and
(d) a DNA which is hybridizable with a DNA having the nucleotide sequence of SEQ ID NO: 3 or a probe that can be produced from said nucleotide sequence under stringent conditions.

3. The DNA according to claim 1, which originates from a chromosome of a Methylophilus bacterium.

4. A methanol-utilizing bacterium, whereby the DNA according to claim 1 has been introduced, and said bacterium has improved ability to produce a polysaccharide.

5. The bacterium according to claim 4, which is a Methylophilus bacterium.

6. A method for producing a polysaccharide, comprising the steps of
A) culturing the bacterium according to claim 4 in a medium containing methanol as a major carbon source, allowing accumulation of the polysaccharide in the medium or in the bacterium, and
B) collecting the polysaccharide from the medium or the cells.

7. A methanol-utilizing bacterium having an ability to reduce production of a polysaccharide, wherein a gene on said bacterium's chromosome has the same nucleotide sequence as the DNA of claim 1, or which has homology to the DNA of claim 1 to such an extent that homologous recombination results in disruption of said DNA, thereby suppressing expression of the gene.

8. The bacterium according to claim 7, which is a Methylophilus bacterium.

9. A method for producing a target substance comprising the steps of
A) culturing the bacterium according to claim 7 which produces the target substance other than polysaccharide in a medium containing methanol as a major carbon source, allowing accumulation of the target substance in the medium or cells of the bacterium and
B) collecting the target substance from the medium or the cells.


SEQUENCE LISTING (13 sequences)

SEQ ID NO: 1 - DNA, 1404 bp, Methylophilus methylotrophus
  CDS (1)..(1404)
atggcgactaaacctccgatcagaacactctccggcttttcatctggcgggagtaatccactttacatgcttgagtctctcgttgagcccttggtgatggtgtttgtgctgtgggggttgtttatttataccgaaaaccgcattccgatgtcgatttttattacatcgatagtgctgttttcgatttctttccccagcggcgccaagattcgcaagggctttgccaagatgtgccgggatgtgattggtcaatggctggtcattgccacctttttgctgacctttgcttatatcactcgttacatcaccttatatagcgaaaaattaattctcgcctggttgattgtgacgccagttgcccagattattgcgttgcagttactaaaatgggccagccccaaattgattgagtggcaaggaccacgacaaaacaccttgattatcggcttgaatgagcaaggtctgcttttggcggataatctgaaacgtgattattatcaaagaatcaatatattgggattttttgaggaccgcgcgcctaaccggcttccgcacatagattcttatccggtacttggcagcttgaatgaactgagtcattacctgaaatcacacactgtacacaaactttatatcgctttaccgatgtccagtcaccctcgtattttgaaactattagacgatcttaaagacacgacagcttccatttactttgtgcctgacatctttgtcaccgacctgatccagggacgcgtttcggatgtcaacggcattcctgttgtttctgtgtgtgatacgccatttactggcatggatggctttatcaaacgcacggcagatattttattttcattattggtgttgattctgatctcgcctattttgatcggtattgcgattgcagtaaaactcacctctcctggccccgttattttcaagcaacgtcgttacggcttggatggacaacagattttggtgtacaagttccgctccatgaccgtcactgaagatggtgcaacggtgacacaagccaccaggaatgatcaacgcattacgccactgggtgcctttttgcgcaaaacctccctggatgagttgccgcagtttattaatgtgttacaaggccgcatgagtgtggttgggccacgcccacatgcggtggcgcataacgaggaataccgtaagctgattaaaggctatatggtacgccacaaggtaaaacccgggattaccggctgggcacaggtaaatggcttccgcggcgaaacggacacgttagaaaaaatggagcaacgtgtccattatgaccttgagtacctgcgcaactggagccctcgcttggatatgttgattgtcgccaagacgatatggctgaccattgttggtcaagatggggcttattag

SEQ ID NO: 2 - protein, 467 aa, Methylophilus methylotrophus
MATKPPIRTLSGFSSGGSNPLYMLESLVEPLVMVFVLWGLFIYTENRIPMSIFITSIVLFSISFPSGAKIRKGFAKMCRDVIGQWLVIATFLLTFAYITRYITLYSEKLILAWLIVTPVAQIIALQLLKWASPKLIEWQGPRQNTLIIGLNEQGLLLADNLKRDYYQRINILGFFEDRAPNRLPHIDSYPVLGSLNELSHYLKSHTVHKLYIALPMSSHPRILKLLDDLKDTTASIYFVPDIFVTDLIQGRVSDVNGIPVVSVCDTPFTGMDGFIKRTADILFSLLVLILISPILIGIAIAVKLTSPGPVIFKQRRYGLDGQQILVYKFRSMTVTEDGATVTQATRNDQRITPLGAFLRKTSLDELPQFINVLQGRMSVVGPRPHAVAHNEEYRKLIKGYMVRHKVKPGITGWAQVNGFRGETDTLEKMEQRVHYDLEYLRNWSPRLDMLIVAKTIWLTIVGQDGAY

SEQ ID NO: 3 - DNA, 1422 bp, Methylophilus methylotrophus
  CDS (1)..(1422)
atgtctttaatgaaaattgtccccgtcattttgtccggtggttctggtacgcgattatggccgttgtcacgcgcggttttgcctaaacagttattgcctttggtgaccgaaaatacgatgttacaggagacattgatccggctttctagctgggcggatgtcggtcatcctatcgtcgtctgtggtaacgatcatcgctttttggtggcggagcaattacggcaagtgaatttgacacctgaggcgattgtgctggagccggtggcgcgaaatacggcacctgcgattgctgctgcggctgtgactttaaaagacaaagatgtcttgatgctggtgttgcctgcggatcatgtgattactgacgtcactgcttttgaggctgctgtgcgtcgtgcctgcgttgcagcagagcaggggaaactggtcacatttggtatagagcctacacagccggaaaccggttatggttatatccaatcaggtgcagaattggaagcatgtgatggttgctttgaagtggcacgttttgttgagaagcctgatgctgcgactgcacagcaatatttggatgccggaaacttttattggaacagcggcatgtttttgtttaaaccggctgtgttcctggctgagttgcagcaatacgcgccagccatggtcagtgcggtaagcaatgccgttgcgcaaagttataaagacctggattttgtgcgcttgcatgaggcctcgtttgctgagtctccttctgattcaattgactatgccgtcatggaaaaaaccaaactggcggccgtggtacctgccagcatggggtggaatgatgttggctcatggactgccttaaaagaagtgcagcccaatgatgcggatgggaatgctacacgcggggatgtgtttcttaaaaatgtgaaaaataccttggtacgggcggaagagcgctttgtggctgccgttggcgtagaggatttgctgattgttgaaaccagtgatgcgatcctggttgcgcaccgtgattgtgcgcaggatgtcaagaatattgttgatcatttgaaggcaagcggacgttctgaacataagatgcatccccgtgtttatcgcccttggggttggtacgagggaatcgatatcggcgagcgtttccaggtcaagcgtattatggtgaaaccaggtgaaagattgtcactgcaaatgcatcatcatcgggctgagcactgggtggttgtcagtgggtctgccatgatcactattgatgatgtcaccaagctctatactgaaaacgaatctacttatataccgattggctcaacgcaccgactagagaatccaggtaaattgcctttgcatttaatcgaggtgcaatccggtagttatcttggagaagatgacatcgtgcgttttgaagatacctacggccgtagttag

SEQ ID NO: 4 - protein, 473 aa, Methylophilus methylotrophus
MSLMKIVPVILSGGSGTRLWPLSRAVLPKQLLPLVTENTMLQETLIRLSSWADVGHPIVVCGNDHRFLVAEQLRQVNLTPEAIVLEPVARNTAPAIAAAAVTLKDKDVLMLVLPADHVITDVTAFEAAVRRACVAAEQGKLVTFGIEPTQPETGYGYIQSGAELEACDGCFEVARFVEKPDAATAQQYLDAGNFYWNSGMFLFKPAVFLAELQQYAPAMVSAVSNAVAQSYKDLDFVRLHEASFAESPSDSIDYAVMEKTKLAAVVPASMGWNDVGSWTALKEVQPNDADGNATRGDVFLKNVKNTLVRAEERFVAAVGVEDLLIVETSDAILVAHRDCAQDVKNIVDHLKASGRSEHKMHPRVYRPWGWYEGIDIGERFQVKRIMVKPGERLSLQMHHHRAEHWVVVSGSAMITIDDVTKLYTENESTYIPIGSTHRLENPGKLPLHLIEVQSGSYLGEDDIVRFEDTYGRS

SEQ ID NO: 5 - DNA, 33 bp, Artificial Sequence
  Description of Artificial Sequence primer MgtfA-F1
ctgagtttgcttgcctattggatcactgctgcc

SEQ ID NO: 6 - DNA, 33 bp, Artificial Sequence
  Description of Artificial Sequence primer MgtfA-R1
cgccaaaattcacaccaccgattctcagcgcat

SEQ ID NO: 7 - DNA, 45 bp, Artificial Sequence
  Description of Artificial Sequence primer Km4-F2
cttgatatcgctagctcgtatgttgtgtggaattgtgagcggata

SEQ ID NO: 8 - DNA, 39 bp, Artificial Sequence
  Description of Artificial Sequence primer Km4-R2
accaacgcgtaatcgccccatcatccagccagaaagtga

SEQ ID NO: 9 - DNA, 35 bp, Artificial Sequence
  Description of Artificial Sequence primer Km4-R1
ttggtgattttgaacttttgctttgccacggaacg

SEQ ID NO: 10 - DNA, 27 bp, Artificial Sequence
  Description of Artificial Sequence primer mManC-F1
ccggatccgatgcgtgtgcctttagtc

SEQ ID NO: 11 - DNA, 28 bp, Artificial Sequence
  Description of Artificial Sequence primer mManC-R1
ccggatcccacctaactacggccgtagg

SEQ ID NO: 12 - DNA, 33 bp, Artificial Sequence
  Description of Artificial Sequence primer mManC-F2
atttgaggtcggtttgcttgcgctattttaacg

SEQ ID NO: 13 - DNA, 31 bp, Artificial Sequence
  Description of Artificial Sequence primer mManC-R2
tcgtgacatagcgttgcacatagccctcata